(19) United States
(12) Patent Application Publication
(10) Pub. No.: US 2020/0046285 A1
(43) Pub. Date: Feb. 13, 2020

(54) DETECTION OF A SIGN OF COGNITIVE DECLINE FOCUSING ON CHANGE IN TOPIC SIMILARITY OVER CONVERSATIONS

(71) Applicant: INTERNATIONAL BUSINESS MACHINES CORPORATION, Armonk, NY (US)

(72) Inventors: Keita Shimmei, Tokyo (JP); Kaoru Shinkawa, Tokyo (JP); Yasunori Yamada, Saitama (JP)

(21) Appl. No.: 16/058,057

(22) Filed: Aug. 8, 2018

(51) Int. Cl. A61B 5/00; G06N 99/00

(52) U.S. Cl. CPC A61B 5/4803; G06N 99/005; A61B 5/4088; A61B 5/7282; A61B 5/7267; A61B 5/7275; A61B 5/7246

(57) ABSTRACT

A computer-implemented method for supporting detection of a sign of cognitive decline is disclosed. In the method, a reference set of conversational data recorded for an individual and one or more sets of conversational data recorded for the individual on different days from the reference set are obtained. The method includes evaluating at least a temporal separation between conversations corresponding to the reference set and each of the one or more sets of the conversational data determine a value of the temporal separation. The method also includes determining topic similarity between the reference set and each of the one or more sets of the conversational data. A feature is generated for the individual based, at least in part, on relationship between the value and the topic similarity, and the computed feature is then sent as a message corresponding to a diagnosis.

### BACKGROUND

The present disclosure, generally, relates to diagnosis support technology, more particularly, to techniques for supporting a detection of a sign of cognitive decline, which may be associated with dementia due to neurodegenerative diseases such as Alzheimer's disease, etc.

As the worldwide elderly population increases, the incidence of the dementia is becoming an increasingly serious health and social problem. Early diagnosis and intervention have been increasingly recognized as a possible way of improving dementia care.

According to recent advances in digital devices such as tablets, mobile phones, and IoT (Internet of Things) sensors, monitoring technology capable of detecting early signs of dementia in everyday situations has great potential for supporting earlier diagnosis and intervention.

The short-term memory loss associated with dementia makes ordinary conversation difficult because of language dysfunctions such as word-finding and word-retrieval difficulties. These language dysfunctions have typically been characterized by using linguistic features, which typically focus on vocabulary richness, repetitiveness, syntactic complexity, etc. Conventionally, the linguistic features that are extracted from speech data while individuals perform neuropsychological tests have been used to try to estimate the risk of the neurodegenerative diseases and cognitive decline.

However, there is still a need for developing novel technology to improve estimation performance of the risk of the neurodegenerative diseases and the cognitive decline.

### SUMMARY

According to an embodiment of the present invention, a computer-implemented method for supporting detection of a sign of cognitive decline is provided. The method includes obtaining a reference set of conversational data recorded for an individual and one or more sets of conversational data recorded for the individual on different days from the reference set. The method includes calculating a value that evaluates at least a temporal separation between conversations corresponding to the reference set and each of the one or more sets of the conversational data. The method also includes calculating topic similarity between the reference set and each of the one or more sets of the conversational data. The method further includes computing a feature for the individual based, at least in part, on relationship between the value and the topic similarity and outputting the feature computed for the individual.

Computer systems and computer program products relating to one or more aspects of the present invention are also described and claimed herein.

Additional features and advantages are realized through the techniques of the present invention. Other embodiments and aspects of the invention are described in detail herein and are considered a part of the claimed invention.

### BRIEF DESCRIPTION OF THE DRAWINGS

The subject matter, which is regarded as the invention, is particularly pointed out and distinctly claimed in the claims at the conclusion of the specification. The foregoing and other features and advantages of the invention are apparent from the following detailed description taken in conjunction with the accompanying drawings in which:

FIG. 1 illustrates a block diagram of a diagnosis support system for cognitive decline according to an exemplary embodiment of the present invention;

FIG. 2 is a flowchart depicting a process for extracting a novel feature to support a detection of cognitive decline according to an exemplary embodiment of the present invention;

FIG. 3 describes definitions of features used for calculating an evaluation value that evaluates at least a temporal separation between conversations corresponding to a reference document and other document according to the exemplary embodiment of the present invention;

FIG. 4 depicts a schematic of way of processing a conversation document as preprocessing for calculating topic similarity according to a particular embodiment of the present invention;

FIG. 5 depicts a schematic of a graphical model of a LDA (Latent Dirichlet Allocation) topic model according to a particular embodiment of the present invention;

FIG. 6 depicts a schematic of a way of calculating topic similarity between each paired documents through several processes according to the exemplary embodiment of the present invention;

FIG. 7 illustrates a plot of a plurality of data points each consisting of topic similarity and an evaluation value;

FIG. 8 is a flowchart depicting a process for optimizing a parameter of an evaluation function for calculating the novel feature according to an exemplary embodiment of the present invention;

FIG. 9 illustrates a graph of discriminative power in a parameter space (β_{P}−β_{Q}) where other two parameters β_{T}, β_{N} are fixed;

FIG. 10 is a flowchart depicting a process for detecting a sign of cognitive decline for a subject user using the novel feature according to an exemplary embodiment of the present invention; and

FIG. 11 depicts a computer system according to one or more embodiment of the present invention.

### DETAILED DESCRIPTION

Hereinafter, the present invention will be described with respect to particular embodiments, but it will be understood by those skilled in the art that the embodiments described below are mentioned only by way of examples and are not intended to limit the scope of the present invention.

One or more embodiments according to the present invention are directed to computer-implemented methods, computer systems and computer program products for supporting detection of a sign of cognitive decline, in which a novel feature that characterizes change in topic similarity over conversations on different days of an individual is computed from at least three sets of conversational data recorded for the individual. One or more other embodiments according to the present invention may be directed to computer-implemented methods, computer systems and computer program products for evaluating a change in topic similarity over conversations on different days of an individual to support detection of the sign of the cognitive decline, in which a novel evaluation value that evaluates a temporal separation between conversations and an amount of speeches in the conversations and that can be used to evaluate the change in the topic similarity over the conversations is calculated for a pair of sets of conversational data of the individual.

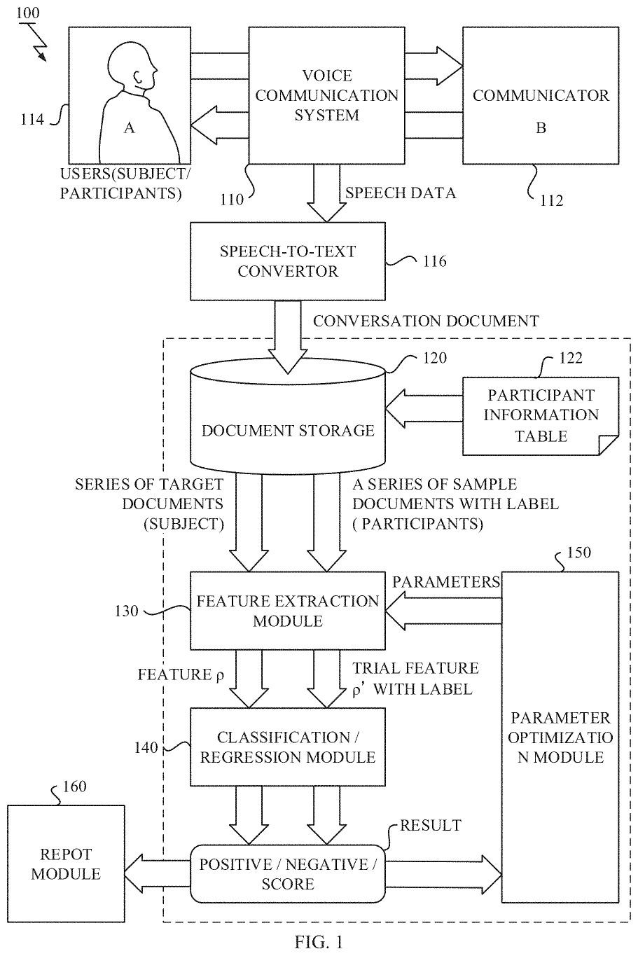

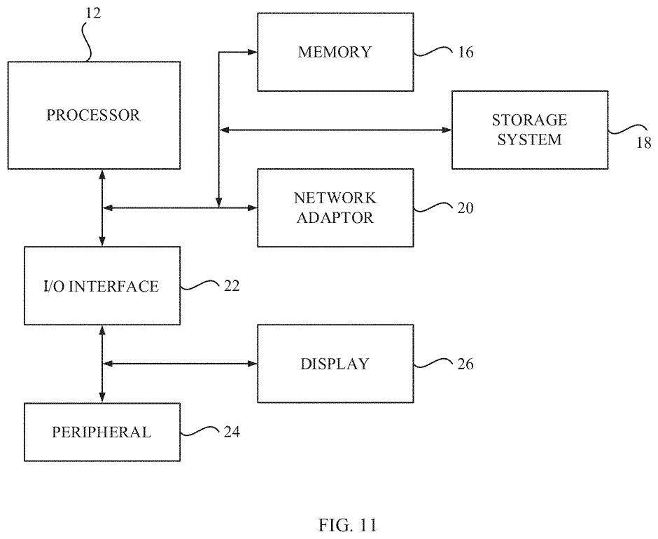

Hereinafter, referring to a series of FIGS. 1-10, a computer system and processes for supporting detection of a sign of cognitive decline according to an exemplary embodiment of the present invention, in which a novel feature that characterizes change in topic similarity over conversations is computed by calculating topic similarity and a novel evaluation value that evaluates a temporal separation between conversations and an amount of speeches in the conversations, will be described. Then, experimental studies according to the exemplary embodiment of the present invention will be described. Finally, referring to FIG. 11, a hardware configuration of a computer system according to one or more embodiments of the present invention will be described.

### Exemplary Embodiment

Hereinafter, with reference to a FIG. 1, a diagnosis support system for supporting detection of an early sing of cognitive decline, which may be associated with dementia due to neurodegenerative diseases such as, Alzheimer's disease, Parkinson's disease, etc., is described.

FIG. 1 illustrates a block diagram of a diagnosis support system 100 for cognitive decline. As shown in FIG. 1, the diagnosis support system 100 may include a voice communication system 110 that mediates the exchange of the voice communications between a communicator 112 and a user 114; a speech-to-text convertor 116 that converts speech signal transmitted through the voice communication system 110 into a text; and a document storage 120 that stores a text transcribed by the speech-to-text convertor 116 as a conversation document, which is a set of conversational data recorded for the user 114 during a conversation with the communicator 112.

The voice communication system 110 may be any one of known systems that can mediate the exchange of at least voice communications between at least two parties (e.g., the communicator 112 and the user 114). Such system may include a telephone exchange system, a VoIP (Voice over Internet Protocol) phone system, a voice chat system and a video call system, to name but a few. Note that the voice communication system 110 is schematically depicted in FIG. 1 as one box: however, the voice communication system 110 may include facilities, cables, devices, etc., which may include terminal devices for the two parties such as a feature phone, a smart phone, a tablet computer, a smart speaker device, etc.

The user 114 may be a subject who is a target individual of detection of early signs of the cognitive declines or a participant who participates in contributions to improve the detection performance of the system 100, according to a registration of the user 114 to the diagnosis support system 100. The user 114 may be registered as either of the subject (a recipient of diagnosis support service whose healthy status is unknown) and the participant (e.g., a healthy control or a patient), or as both (a recipient of the service who is currently considered healthy).

The information of the participants is managed in a participant information table 122. When registering to the system or updating the user information in the system, the participant or his/her family may report whether he/she is suffering from cognitive decline or is diagnosed as being healthy. Furthermore, the family may report the severity of the cognitive decline when the participant is suffering from the cognitive decline. The participant information table 122 may hold, for each participant, a label indicating whether the participant is reported as a healthy control or a patient. In a preferable embodiment, the participant information table 122 may further include severity information for each participant who is suffering from the cognitive decline.

The communicator 112 may be a human communicator (e.g., a social worker or an staff of a service provider) or a family member of the user 114 who may call the user 114 on a regular or occasional basis to have a daily conversation for certain period such as several minutes. Alternatively, the communicator 112 may be a computational system such as a voice chat bot or a social robot that can mimic a human communicator.

The speech-to-text convertor 116 is configured to convert to a text from speech signal that is transferred from the voice communication system 110. In a particular embodiment, the speech signal of both the user 114 and the communicator 112 may be transferred to the speech-to-text convertor 116. Each text transcribed from the speech signal that is recorded during a single conversation is stored in the document storage 120 as a conversation document in association with identification information (ID) of the user 114 and timestamp (or dates). The conversation document recorded for the participant may be stored as sample conversation documents in further association with a label regarding the cognitive decline, which may be obtained from the participant information table 122. Speaker of each speech or utterance may be discriminated on the basis of channel or speaker identification/diarization techniques.

In the embodiment, it is described that the voice communication system 110 is used to acquire speech signals by intervening in the remote voice communication between the user 114 and the communicator 112. However, the way of acquiring the speech signals between the user 114 and the communicator 112 is not limited to the specific way. In other embodiments, instead of using the voice communication system 110 that mediates the exchange of the remote voice communication, there may be an apparatus such as a smart speaker device and a recording device that can acquire sound signal from the surrounding environment where the user 114 and the communicator 112 perform face-to-face conversations in everyday life situations. In such case, the speaker of each speech or utterance can be discriminated by the speaker identification/diarization techniques and transferred to the speech-to-text convertor 116 with speaker information via a network or a removable media.

Referring further to FIG. 1, the diagnosis support system 100 includes a feature extraction module 130 that performs novel feature extraction according to the exemplary embodiment; a classification/regression module 140 that infers a health state of the user 114 based on a result output from the feature extraction module 130.

The feature extraction module 130 is configured to compute a novel feature that characterizes a change (or transition) in topic similarity over day-to-day conversations based, at least in part, on a series of conversation documents recorded for the same user 114. The series of the conversation documents may include one conversation document Dᵢ picked up as a reference document and a set of one or more conversation documents {Dⱼ} satisfying a predetermined condition with respect to the reference document Dᵢ.

While computing one value of the novel feature, the reference document Dᵢ may be fixed. The set of the conversation documents {Dⱼ} may include a plurality of documents recorded on different days from the reference document Dᵢ. The predetermined condition may be a condition for searching conversation documents of the same user 114 whose time difference with respect to the reference document Dᵢ is within a predetermined period.

To compute the novel feature, the feature extraction module 130 is configured to calculate a novel evaluation value Sᵢⱼ that evaluates at least a temporal separation between conversations corresponding to the reference document Dᵢ and each element in the set of the conversation documents {Dⱼ}. Note that the temporal separation means a degree of separation (or simply a period of time) between first and second conversations along with time axis (e.g., representing the passage of days, the course of day-to-day conversations). In the described embodiment, the novel evaluation value Sᵢⱼ further evaluates an amount of speeches in the conversations corresponding to the reference document Dᵢ and each element in the set of the conversation documents {Dⱼ}. To calculate the novel evaluation value Sᵢⱼ, the feature extraction module 130 uses one or more parameters, which will be described in more detail later.

To compute the novel feature, the feature extraction module 130 is further configured to calculate topic similarity Yᵢⱼ between the reference documents Dᵢ and each element in the set of the conversation documents {Dⱼ}. In a particular embodiment, the topic similarity can be calculated based, at least in part, on Latent Dirichlet Allocation (LDA), where a set of topics are extracted from each of the conversation documents Dᵢ, {Dⱼ} and the topic similarity between two documents Dᵢ, Dⱼ can be calculated based on the extracted sets of the topics for the two documents Dᵢ, Dⱼ. In a particular embodiment, the topic similarity may be measured as cosine similarity, which measures cosine of an angle between vectors representing the reference documents Dᵢ and each element in the conversation document set {Dⱼ}. More detail about the topic similarity calculation will be described later.

After obtaining a plurality of data points (Sᵢⱼ, Yᵢⱼ) for all elements in the conversation document set {Dⱼ} with respect to the reference document Dᵢ, the feature extraction module 130 computes a novel feature p for the user 114 based, at least in part, on statistical relationship between the evaluation value Sᵢⱼ and the topic similarity Yᵢⱼ and outputs the feature ρ to the subsequent module, i.e., the classification/regression module 140. In a particular embodiment, the feature ρ is a correlation coefficient, which is a measure of correlation between plural variables, between the topic similarity Yᵢⱼ and the evaluation value Sᵢⱼ as the variables. In a further particular embodiment, Pearson correlation coefficient, which is a measure of linear correlation of two variables, can be used as the feature ρ. In other embodiments, a coefficient of linear regression can also be used as the feature ρ. More detail about the feature computation based on the evaluation value Sᵢⱼ and the topic similarity Yᵢⱼ will be described later.

The classification/regression module 140 is configured to infer a health state of the user 114 based on the novel feature ρ extracted by the feature extraction module 130. The classification/regression module 140 may be based on any machine learning models, including a classification model, a regression model, etc.

When the classification/regression module 140 is based on the classification model, the health state inferred by the classification/regression module 140 may be represented by a class indicating whether or not there is any signs of the cognitive decline (e.g., positive/negative for the binary classification) or the degree of the risk of the cognitive decline (e.g., levels of severity (no risk/low risk/high risk) for multinomial classification). When the classification/regression module 140 is based on the regression model, the health state inferred by the classification/regression module 140 may be represented as a value that measures the degree of the risk of the cognitive decline (e.g., severity score). Depending on the granularity of the inference requested, appropriate label information would be prepared for each sample conversation document.

In a particular embodiment, to infer the health state of the user 114, the classification/regression module 140 can utilize the feature ρ extracted by the feature extraction module 130 solely or in combination with one or more other features. Such other feature may be any of known features including, but not limited to, features relating to vocabulary richness (e.g., type-token ratio (TTR), Brunet's index (BI), and Honore's statistics (HS)), features relating to repetitiveness (e.g., frequency of repeated words and phrases, sentence similarities), features relating to syntactic complexity (e.g., mean length of sentences, “part-of-speech” frequency, and dependency distance).

Referring further to FIG. 1, the diagnosis support system 100 may further include a parameter optimization module 150 that optimizes one or more parameters of the feature extraction module 130 based on results inferred by the classification/regression module 140 with provisional parameters and labels associated with the result; and a report module 160 that reports a result inferred by the classification/regression module 140 with optimized parameters to the user 114 or his/her family via appropriate communication tool such as e-mail, instant message, web site, mobile application, etc.

The diagnosis support system 100 may have multiple modes of operation, including a learning mode where the parameter optimization module 150 works and an inference mode where the report module 160 operates.

First, operations in the learning mode are described with reference further to FIG. 1. In the document storage 120, a collection of training samples, each of which includes a plurality of sample conversation documents recorded for a participant user 114 and a label regarding the cognitive decline of the participant user 114, may be prepared. The label may be prepared by using the information managed in the participant information table 122 as described above. The collection of the training samples may include sample conversation documents recorded for a variety of participants.

The feature extraction module 130 is configured to use a given evaluation function that evaluates a temporal separation between the conversations and an amount of speeches in the conversations to compute the feature ρ. In the learning mode, the parameter optimization module 150 is configured to optimize parameters of this evaluation function such that discriminative power of the computed feature ρ is maximized.

The parameter optimization module 150 may pick up one or more series of sample conversation documents that are stored in the document storage 120. Each series of the sample conversation documents may include a reference sample document Dᵢ′ and a set of one or more sample documents {Dᵢ}′ satisfying the predetermined condition, which has been described above.

The parameter optimization module 150 may feed each series of the sample conversation documents (Dᵢ′, {Dᵢ′}) into the feature extraction module 130. The feature extraction module 130 may output, for each series, a trial feature ρ′ calculated using the evaluation function with the current provisional value of the parameters. The classification/regression module 140 may receive the trial feature ρ′ and output a trial result of the inference based on the trial feature ρ′, for each series of the sample conversation documents (Dᵢ′, {Dⱼ′}). The parameter optimization module 150 may receive results of the inference from the classification/regression module 140 and update the parameters of the evaluation function by comparing each result of the inference and each label associated with each series. More detail about the parameter optimization will be described later.

Next, operations in the inference mode are described with reference further to FIG. 1.

In the document storage 120, a series of target documents recorded for a subject user 114 is accumulated.

In the inference mode, the report module 160 may pick up at least one series of target conversation documents of the subject user 114 that are stored in the document storage 120. The series of the target conversation documents may include a reference target document Dᵢ and a set of one or more target documents {Dⱼ} satisfying the predetermined condition.

The report module 160 may feed at least one series of the target documents (Dᵢ, {Dⱼ}) into the feature extraction module 130. The feature extraction module 130 may output a computed feature ρ calculated for the subject user 114 by using the evaluation function with the parameters optimized by the parameter optimization module 150. The classification/regression module 140 may receive the feature ρ and output a result of the inference for the subject user 114 based on the feature ρ. The report module 160 may report the result of the inference provided by the classification/regression module 140 to the user 114 or his/her family via appropriate communication tool.

Note that more than two series of the conversation documents where different documents are selected as respective reference documents can be used to infer the health state of the subject user 114 in order to improve performance and stability of the detection. For example, more than two features calculated from the more than two series of the conversation documents can be subjected to statistical processing (e.g., average) and a statistic of features (e.g., averaged feature) can be used as an input for the classification/regression module 140. For another example, more than two features calculated from the more than two series of the conversation documents can be used as an input for the classification/regression module 140, respectively.

In the described embodiment, the result can be used as diagnosis support data to help medical diagnosis by doctors as screening for example and/or to give a suggestion for the subject user 114 to see a doctor when necessary.

In particular embodiments, each of the modules 110, 116, 120, 122, 130, 140, 150 and 160 in the diagnosis support system 100 described in FIG. 1 may be implemented as a software module including program instructions and/or data structures in conjunction with hardware components such as a processing circuitry (e.g., a CPU (Central Processing Unit), a processing core, a GPU (Graphic Processing Unit), a FPGA (Field Programmable Gate Array)), a memory, etc.; as a hardware module including electronic circuitry (e.g., a neuromorphic chip); or as a combination thereof.

These modules 110, 116, 120, 122, 130, 140, 150 and 160 described in FIG. 1 may be implemented on a single computer system such as a personal computer and a server machine or a computer system distributed over a plurality of computing devices such as a computer cluster of computing nodes, a client-server system, a cloud computing system and an edge computing system. In a particular embodiment, the diagnosis support system 100 according to the exemplary embodiment can provide a diagnosis support service for the cognitive decline through the internet as a cloud service.

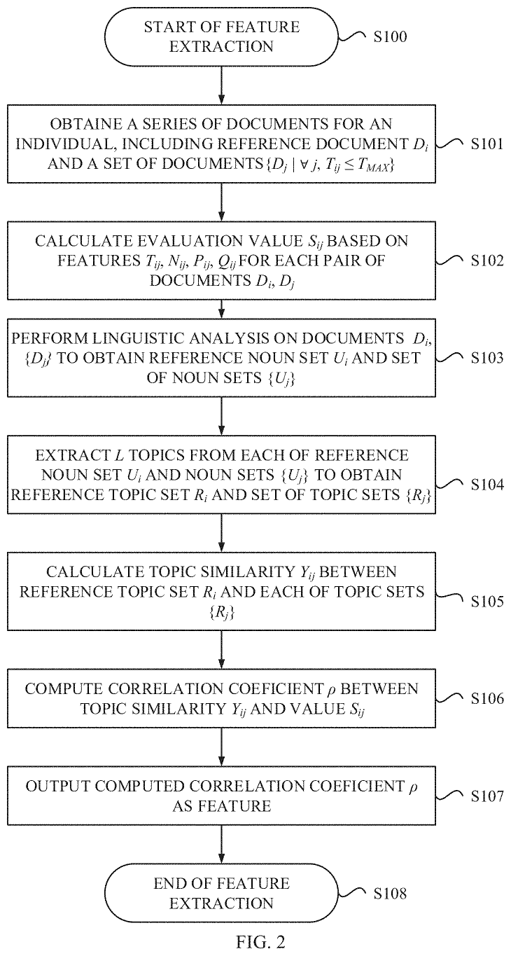

With reference to FIG. 2, a process for extracting a novel feature used to support a detection of cognitive decline according to an exemplary embodiment of the present invention is described. The process may begin at step S100 in response to calling of the process of the feature extraction. Note that the process shown in FIG. 2 may be performed by processing circuitry such as one or more processing units. Also note that the flow of the process shown in FIG. 2 may be common in both the learning mode and the inference mode, except for parameters of the feature extraction module 130.

A step S101, the processing circuitry may obtain a series of conversation documents of a user 114 who is a subject in the inference mode or one of the participants in the learning mode. The series of the conversation documents may include the reference document Dᵢ and a set of one or more conversation documents that satisfies a predetermined condition {Dⱼ|∀j, Tᵢⱼ≤T_{MAX}}, where Tᵢⱼ denotes the number of days between the conversations and T_{MAX} represents an upper limit of the number of days between the conversations to use, which defines a range of documents to be taken into consideration.

At step S102, the processing circuitry may calculate an evaluation value Sᵢⱼ based on features Tᵢⱼ, Nᵢⱼ, Pᵢⱼ, Qᵢⱼ for each pair of the reference document Dᵢ and one of the conversation documents {Dⱼ}. In a particular embodiment, the evaluation value Sᵢⱼ can be calculated as a function h( ) of these features Tᵢⱼ, Nᵢⱼ, Pᵢⱼ, Qᵢⱼ, more specifically, a weighted sum of these features Tᵢⱼ, Nᵢⱼ, Pᵢⱼ, Qᵢⱼ with weights β_{T}, β_{N}, β_{P}, β_{Q}, as follow:

$${S}_{ij} = h ( {T}_{ij} , {N}_{ij} , {P}_{ij} , {Q}_{ij} ) = {β}_{T} \cdot {T}_{ij} + {β}_{N} \cdot {N}_{ij} - {β}_{P} \cdot {P}_{ij} - {β}_{Q} \cdot {Q}_{ij} ,$$

where β−[0,1].

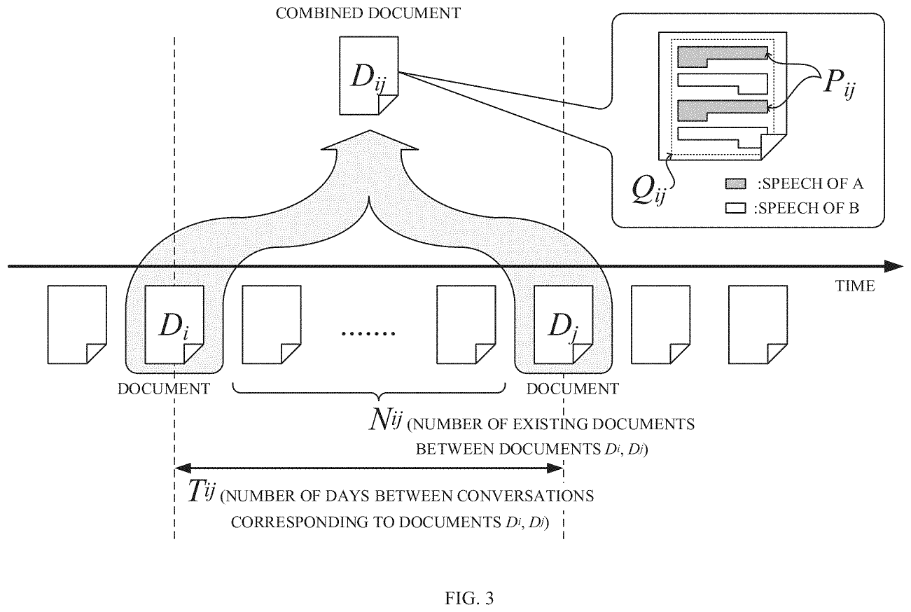

Referring to FIG. 3, definitions of these features Tᵢⱼ, Nᵢⱼ, Pᵢⱼ, Qᵢⱼ used for calculating the evaluation value Sᵢⱼ that evaluates at least the temporal separation between conversations corresponding to the reference document Dᵢ and other document Dⱼ are described.

As shown in FIG. 3, there are a series of conversation documents recorded for an individual along a time axis that represents the passage of days and/or the course of the day-to-day conversations. Among these conversation documents, the reference documents Dᵢ is picked up and other document Dⱼ corresponding to a day different from the reference documents Dᵢ is also picked up to form paired documents (Dᵢ, Dⱼ).

The number of days between the conversations (not including the day of the first conversation but including the day of the second conversation) corresponding to the paired documents (Dᵢ, Dⱼ), Tᵢⱼ, is one of features that evaluate the temporal separation between conversations corresponding to the paired documents (Dᵢ, Dᵢ). Note that the number of the days Tᵢⱼ can be calculated based on the timestamps or the dates associated with the paired conversation documents (Dᵢ, Dᵢ). The number of documents (not including both documents for the first conversation and the second conversation) existing between the paired documents (Dᵢ, Dⱼ), Nᵢⱼ, is also one of the features that evaluate the temporal separation between the paired documents (Dᵢ, Dⱼ).

Note that in the described embodiment, the documents Dⱼ are described to be picked up within a certain period after the reference document Dᵢ (timestamp of Dᵢ<timestamps of Dⱼ). Alternatively, in other embodiments, documents Dⱼ may be picked up within a certain period before the reference document Dᵢ(timestamp of Dᵢ>timestamps of Dⱼ).

Since the amount of the speeches in the conversations may vary for each conversation, features that evaluate an amount of speeches in conversations are preferably defined.

In the described embodiment, the amount of the speeches in the conversations is evaluated as a combined total of the paired documents (Dᵢ, Dⱼ). The paired documents Dᵢ, Dⱼ are first combined to generate a combined conversation document Dᵢⱼ, which is used to evaluate the amount of the speeches in the conversations. The combined conversation document Dᵢⱼ can be created by simply concatenating the paired documents Dᵢ, Dⱼ. In the combined document Dᵢⱼ, there are typically one or more speeches spoken by the user (A) 114, which are illustrated by gray boxes in FIG. 3, and one or more speeches spoken by the communicator (B) 112, which are illustrated by white boxes in FIG. 3.

An amount of speeches spoken by the user (A) 114 in both the reference document Dᵢ and each of the documents {Dⱼ}, Pᵢⱼ, is one of features that evaluate the amount of the speeches in conversations corresponding to the reference document Dᵢ and each of the documents {Dⱼ}. A total amount of speeches in both the reference document Dᵢ and each of the documents {Dⱼ}, including speeches spoken by the user (A) 114 and speeches spoken by the communicator (B) 112, Qᵢⱼ, is also one of features that evaluate the amount of the speeches in the conversations corresponding to the reference document Dᵢ and each of the documents {Dⱼ}. Note that total or individual amount of the speeches can be measured as time length of speeches and/or the number of words in the speeches, regardless of parts of speech, or for a specific part of speech (e.g. nouns). Also note that the communicator 112 (B) is not fixed for all the conversations but it may be different for each conversation.

Note that the way of evaluating the amount of the speeches in the conversations is not limited to as the combined total of the paired documents (Dᵢ, Dᵢ), although the parameters to be optimized can be reduced in such a case. In other embodiments, the amount of the speeches in the conversations may be evaluated for each of the paired documents (Dᵢ, Dᵢ), separately.

Among these features Tᵢⱼ, Nᵢⱼ, Pᵢⱼ, Qᵢⱼ, it is preferable to combine the type of the features evaluating the temporal separation (Tᵢⱼ and/or Nᵢⱼ) and the type of features evaluating the amount of the speeches in the conversations (Pᵢⱼ and/or Qᵢⱼ).

Since the type of the features evaluating the amount of the speeches (Pᵢⱼ and/or Qᵢⱼ) may have opposite effect from the type of the features evaluating the temporal separation (Tᵢⱼ and/or Nᵢⱼ), signs for the weights β_{P}, β_{Q} may be opposite to the weights β_{T}, β_{N}.

The evaluation value Sᵢⱼ calculated for one pair of documents Dᵢ, Dⱼ based on these features Tᵢⱼ, Nᵢⱼ, Pᵢⱼ and/or Qᵢⱼ can be used to evaluate the change in the topic similarity over conversations together with other evaluation values calculated for other pairs combined with the reference document Dᵢ.

Referring back to FIG. 2, at steps from S103 to S105, the processing circuitry may calculate the topic similarity Yᵢⱼ between each paired document Dᵢ, Dⱼ.

More specifically, at step S103, the processing circuitry may perform linguistic analysis on each of the reference documents Dᵢ and the documents {Dⱼ} to obtain an reference noun set U; for the reference documents Dᵢ and a set of noun sets {Uⱼ} for the set of the documents {Dⱼ}.

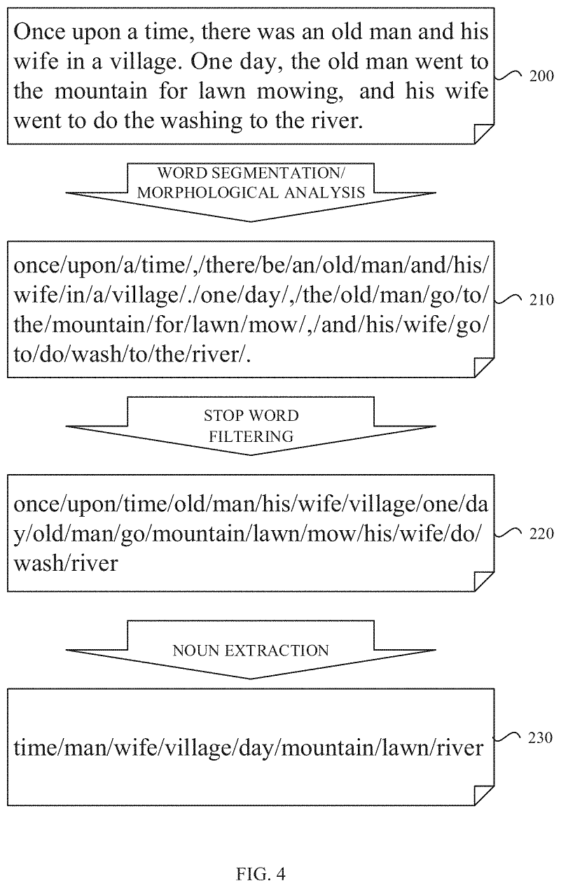

Referring to FIG. 4, a schematic of way of processing a conversation document is depicted as preprocessing for calculating the topic similarity Yᵢⱼ according to a particular embodiment.

Initially, there is an original conversation document 200 that includes one or more sentences, each of which is spoken by either the user 114 or the communicator 112 during a single conversation between the user 114 and the communicator 112. Note that the single conversation does not mean a couple of talks consisting simply of a question and a reply. The single conversation includes, but is not limited to, a series of talks starting with a greeting of hello and ending with greeting of goodbye, for example. Note that example shown in FIG. 4 contains sample sentences in English, for the convenience of description, which has no connection with actual conversation.

In the linguistic analysis at step S103 in FIG. 2, at first, word segmentation/morphological analysis is performed on the original conversation document 200. In the case of language categorized in agglutinative languages such as Japanese, morphological analysis may be performed in order to segment a sentence into words. On the other hand, in the case of English and other languages that have a trivial word delimiter such as space, an original sentence is simply divided by the word delimiter to generate a series of separated words. In addition to the word segmentation, lemmatization may also be performed. After the word segmentation/morphological analysis, a segmented conversation document 210, including a series of separated words, is obtained.

Then, the segmented conversation document 210 is subjected to filtering to remove futile words. Such filtering may include a stop word filtering and a part-of-speech filtering. The stop word filtering is performed to remove specific stop words that are considered preferable to be excluded from processing for reasons as being general. After the stop word filtering, a first filtered conversation document 220 is obtained. The part of speech filtering is performed to remove words categorized into specific parts-of-speech (i.e., parts of speech other than nouns in the described embodiment) and extract words that are categorized into other parts-of-speech (i.e., nouns in the described embodiment). After the part of speech filtering, there is a second filtered conversation document 230, from which the noun set Uᵢ/Uⱼ is obtained finally.

Referring back to FIG. 2, at step S104, the processing circuitry may extract L topics from each of the reference noun set Uᵢ and the noun sets {Uⱼ} to obtain a reference topic set Rᵢ for the reference documents Dᵢ and a set of topic sets {Rⱼ} for the set of the documents {Dⱼ}. In a particular embodiment, the extraction of the topics from each of the noun sets Uᵢ, {Uⱼ} can be done based on Latent Dirichlet Allocation (LDA).

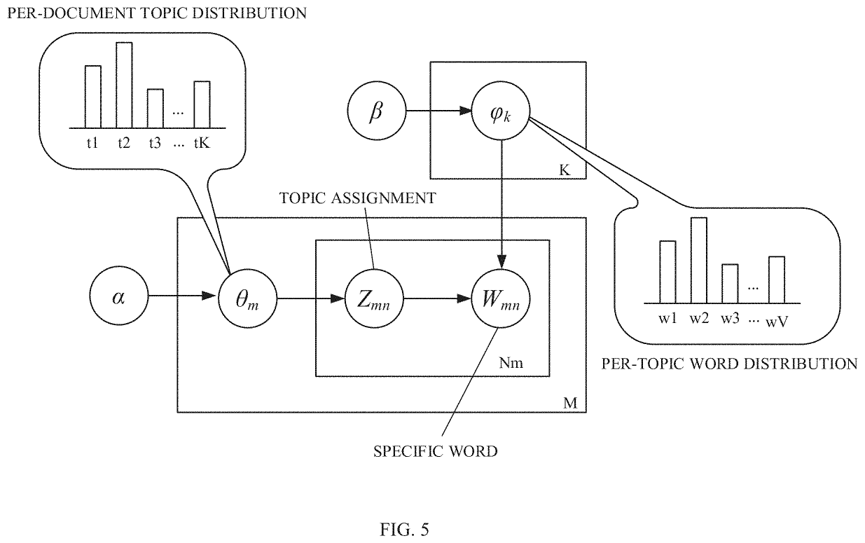

FIG. 5 depicts a schematic of a graphical model of a LDA topic model according to a particular embodiment of the present invention. The LDA topic model is a generative probabilistic model where each document is assumed as a mixture of a small number of topics and creation of each word is assumed to be attributable to one of the topics of the document (David M. Blei, et al., “Latent Dirichlet Allocation”, Journal of Machine Learning Research, 3 (4-5), pp. 993-1022, January 2003).

In the LDA topic model, there is a corpus D of documents. M denotes the number of documents in the corpus D. The document m has a number of words wₘₙ, each of which is located at corresponding positions n. The Nₘ represents the number of words in each document m. A plurality of topics (k=1, . . . , K) is defined in the LDA topic model. There is topic assignment zₘₙ for the n-th word in the document m. θₘ represents topic distribution for the document m. φₖ represents word distribution for the topic k. α is a parameter of the Dirichlet prior on the per-document topic distribution θₘ. β is the parameter of the Dirichlet prior on the per-topic word distribution φₖ.

The parameters of the LDA topic model may be updated by appropriate algorithm such as EM (expectation-maximization) algorithm, Gibbs sampling, etc., with a given corpus D. By using the LDA topic model, topic distribution can be calculated for each document consisting of a set of words. L topics (t₁ₛₜ, t_{2nd}, . . . t_{Lth}) are extracted for each document (i.e., each of noun sets Uᵢ,{Uⱼ}) and each of the reference topic set Rᵢ and the topic sets {Rⱼ} is composed of a L vectors each including noun and word probability of each noun. In one embodiment, L topic vectors may be extracted as whole of the total K topics (i.e., L=K). In other embodiments, L topic vectors may be extracted as a part (top L topics) in the total K topics (i.e., L<K). Also note that each topic vector may be composed of a part of words (e.g., 20 words) having a higher word probability in the whole vocabulary.

A specific way of extracting the topics from each document (i.e., each of the noun sets Uᵢ,{Uⱼ}) based on the LDA is not limited. In one embodiment, L topics are extracted for each document (i.e., each of the noun sets Uᵢ,{Uⱼ}) by giving each document as the corpus D for estimating the LDA topic model. In other embodiments, L topics are extracted for each document (i.e., each of the noun sets Uᵢ,{Uⱼ}) by giving a collection of documents (i.e., a collection of the noun sets Uᵢ,{Uⱼ} picked up for the specific reference document Dᵢ or a collection of whole noun sets Uₓ regardless of the specific reference document Dᵢ) as the corpus D for estimating the LDA topic model. In further other embodiments, the LDA topic model is trained by using an external corpus D_{EXT} in advance and L topics are inferred for each document (i.e., each of the noun sets Uᵢ,{Uⱼ}) by giving each document as an unseen document into the trained LDA topic model.

Also note that in the exemplary embodiment, it is described that the LDA is used to extract the topics from the noun sets Uᵢ, {Uⱼ} that are obtained from the conversation documents Dᵢ, {Dⱼ} with appropriate linguistic analysis; however, topic model is not limited to the LDA. In other embodiments, other topic models including, but not limited to, Latent Semantic Analysis (LSA), Probabilistic Latent Semantic Analysis (PLSA), Non-negative Matrix Factorization (NMF), may also be used to extract topics from the conversation documents Dᵢ, {Dⱼ}. Also note that in the exemplary embodiment, it is described that a set of noun is extracted from the conversation document through appropriate linguistic analysis before topic extraction, the way of extracting the topics from the conversation document is not limited to such a way.

It is described that the processing of the steps S103 and S104 is performed for each time a series of the conversation documents Dᵢ, {Dⱼ} specified by the picked up documents Dᵢ is given. However, the way of obtaining Rᵢ, {Rⱼ} is not limited. Alternatively, in other embodiments, to avoid duplication of calculations, the processing of the steps S103 and S104 may be performed in advance for every document Dₓ in the available document collection.

At step S105, the processing circuitry may calculate topic similarity Yᵢⱼ between the reference topic set Rᵢ and each of the topic sets {Rⱼ}). The topic similarity may be measured as cosine similarity, which measures cosine of an angle between vectors representing the reference documents Dᵢ and each element in the set of the conversation documents {Dⱼ}. Note that in the embodiment where L topics are extracted for each document (Dᵢ or Dⱼ), there are L vectors representing each document (Dᵢ or Dⱼ). The way of calculating the value of the topic similarity Yᵢⱼ for the paired document (Dᵢ, Dⱼ) based on extracted L vectors is not limited. In a particular embodiment, average or maximum of cosine similarities between vectors in all combination of L vectors for Dᵢ and L vectors for Dⱼ (L×L similarities) can be used as the value of the topic similarity Yᵢⱼ.

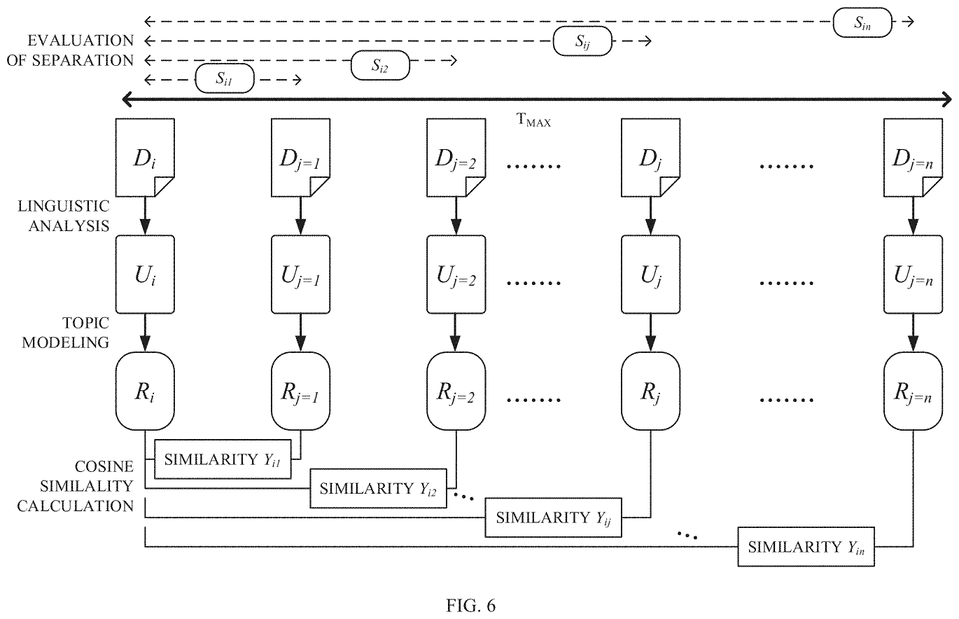

Through the processing of steps from S103 to S105, the topic similarity Yᵢⱼ is calculated for each pair of the reference document Dᵢ and other documents {Dⱼ}. FIG. 6 depicts a schematic of a way of calculating the topic similarity Yᵢⱼ between each paired documents (Dᵢ, Dⱼ) through the processes of steps S103-S105 in FIG. 2. As shown in FIG. 6, for each pair of the reference document Dᵢ and the documents {Dⱼ|∀j, Tᵢⱼ≤T_{MAX}}, the topic similarities Yᵢⱼ are calculated through linguistic analysis, the topic modeling and cosine similarity calculation processes. Furthermore, for each pair of the reference document Dᵢ and the document {Dⱼ|∀j, Tᵢⱼ≤T_{MAX}}, the evaluation value Sᵢⱼ are calculated at step S102 by using the evaluation function together with the features Tᵢⱼ, Nᵢⱼ, Pᵢⱼ, Qᵢⱼ.

Referring back to FIG. 2, at step S106, the processing circuitry may compute a correlation coefficient ρ between the topic similarities Yᵢⱼ and the evaluation values Sᵢⱼ using a plurality of obtained data points (Sᵢⱼ, Yᵢⱼ) for all elements in the conversation document set {Dⱼ} with respect to the fixed reference document Dᵢ.

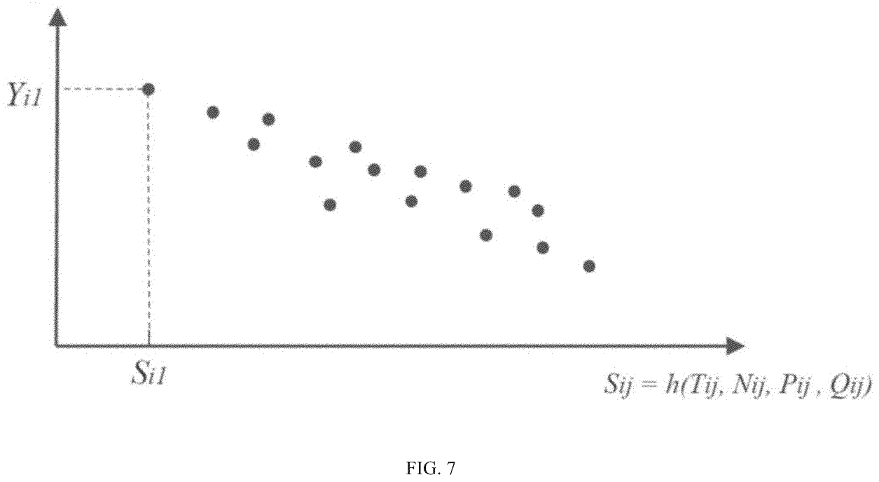

FIG. 7 illustrates a plot of the plurality of the data points, each of which is represented by value of the topic similarity Yᵢⱼ and the evaluation value Sᵢⱼ. As shown in FIG. 7, there may be a statistical relationship between the topic similarity Yᵢⱼ and the evaluation value Sᵢⱼ in a certain case.

When the cognitive function is normal, even though peoples may talk about the same topics as today after one or two days, however, the possibility that the same topic will rise would decrease as they repeat the conversations. Thus, it is considered that the similarity between topics picked up in a conversation on a certain day and topics for another day would be high at the beginning, but, it gradually declines as the days go on. Thus, the topic similarity Yᵢⱼ would decline as the evaluation value Sᵢⱼ that evaluates at least the temporal separation between the conversations corresponding to the reference document Dᵢ and other document Dⱼ becomes larger when the cognitive function is normal. Thus, the correlation coefficient increases in the negative direction.

On the other hand, in case of a person suffering from the cognitive decline, since it is a possible that the people may have forgotten the topic that they talked earlier, there may be no dependency between the topics of previous conversation and the topics of next conversation. Thus, less significant decrease of the topic similarity Yᵢⱼ due to the evaluation value Sᵢⱼ would be observed in comparison to the case where the cognitive function is normal. Thus, the correlation coefficient does not increase in the negative direction.

In the particular embodiment where Pearson correlation coefficient is employed, the correlation coefficient ρ between the topic similarities Yᵢⱼ and the evaluation values Sᵢⱼ can be calculated by following equation:

$$ρ = \frac{{\sum }_{j} ( {S}_{ij} - {\overline{S}}_{ι} ) ( {Y}_{ij} - {\overline{Y}}_{i} )}{\sqrt{{\sum }_{j} {( {S}_{ij} - {\overline{S}}_{ι} )}^{2}} \sqrt{{\sum }_{j} {( {Y}_{ij} - {\overline{Y}}_{ι} )}^{2}}} ,$$

where S̅ₗ̅ and Y̅ᵢ are the means of S and Y, respectively.

At step S107, the processing circuitry may output the computed correlation coefficient ρ as the feature and the process may end at step S108.

In the learning mode, the feature ρ calculated for one participant user 114 according to the process shown in FIG. 2 can be used as an input for the classification/regression module 140 solely or in combination with other feature to compare the inference result with the label of the participant for answer matching. In the inference mode, the feature ρ calculated for one subject user 114 according to the process shown in FIG. 2 can be used as an input for a machine learning model (e.g., classification/regression module 140 or other machine learning model) solely or in combination with other feature to infer whether or not there is any signs of the cognitive decline for the subject, or the degree of a risk of the cognitive decline for the subject.

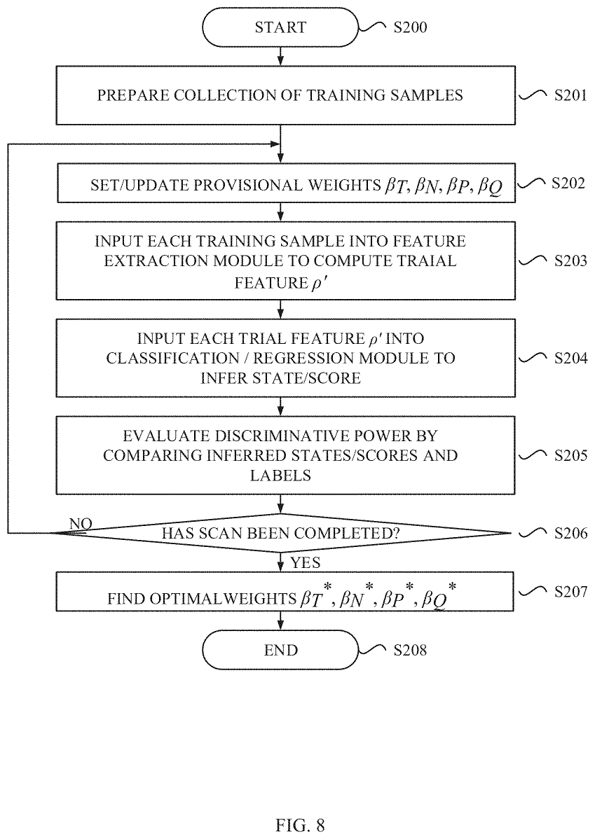

With reference to FIG. 8, a process for optimizing parameters of an evaluation function h( ) that is used for calculating the novel feature ρ according to an exemplary embodiment of the present invention is described. As shown in FIG. 8, the process may begin at step S200 in response to a request of initiating a learning process from an operator. Note that the process shown in FIG. 8 may be performed by processing circuitry such as one or more processing units.

A step S201, the processing circuitry may prepare a collection of training samples, each of which includes one or more sample conversation documents of a corresponding participant with a label associated with the corresponding participant. Each training sample n includes a reference sample document Dᵢₙ and a set of one or more sample documents {Dⱼₙ}.

During the loop from the step S202 to step S206, the weights β_{T}′, β_{N}′, β_{P}′, β_{Q}′ are varied to calculate trial results of the feature ρ′ for every provisional values of the weights β_{T}′, β_{N}′, β_{P}′, β_{Q}′.

At step S202, the processing circuitry may set a provisional value of the weights β_{T}′, β_{N′}, β_{P′}, β_{Q7}. In a particular embodiment, each of the provisional weights β_{T}′, β_{N}′, β_{P}′, β_{Q}′ may be varied from 0 to 1 during the scanning.

At step S203, the processing circuitry may input each training sample (Dᵢₙ, {Dⱼₙ}) into the feature extraction module 130 to compute the trial feature ρₙ′ for each training sample (Dᵢₙ, {Dⱼₙ}).

In the process shown in step S203, the trial result of the feature ρₙ′ is computed from the topic similarity Yᵢₙⱼₙ′ and the evaluation value Sᵢₙⱼₙ′ that is calculated under a current version of the evaluation function characterized by the provisional weights β_{T}′, β_{N}′, β_{P}′, β_{Q}′.

At step S204, the processing circuitry may input each computed trial feature ρₙ′ into the classification/regression module 140 to infer the state/score of the cognitive decline for each training sample n. In a particular embodiment with binary classification, appropriate cut off value is set for each inference. At step S205, the processing circuitry may evaluate discriminative power by comparing each inferred state/score and a corresponding label for all training samples. In a particular embodiment with binary classification, ROC (Receiver Operator Curve)-AUC (Area Under the Curve) and/or effect size can be used to evaluate the discriminative power.

At step S206, the processing circuitry may determine whether or not the scanning of trial weights β_{T}′, β_{N}′, β_{P}′, β_{Q}′ has been completed. If all weights β_{T}′, β_{N}′, β_{P}′, β_{Q}′ has been varied from 0 to 1, for example, the scanning is determined to be completed. In response to determining that the scanning of the weights has not been completed yet (S206: NO), the process may loop back to step S202 for another trial. On the other hand, in response to determining that the scanning of the weights has completed (S202: YES), the process may proceed to step S207.

A step S207, the processing circuitry may find values of weights β_{T}*, β_{N}*, β_{P}*, β_{Q}* that show highest discriminative power as an optimal value and the process may end at step S208. The parameters of the feature extraction module 130 are updated to the optimal one according to the process shown in FIG. 8.

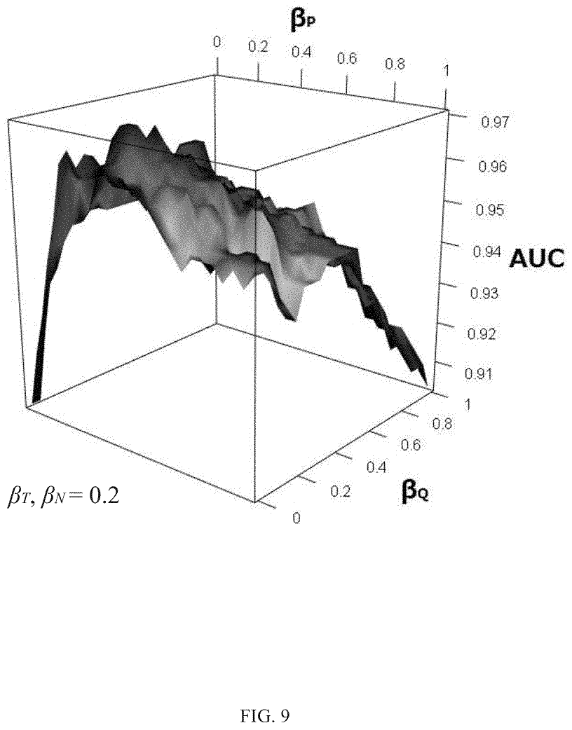

FIG. 9 illustrates a graph of a discriminative power in a parameter space (β_{P}−β_{Q}) where other two parameters β_{T}, β_{N} are fixed. As shown in FIG. 9, the processing circuitry may search a point in the parameter space that maximizes discriminative power.

Note that in the exemplary embodiment grid search approach where the discriminative power is evaluated for every grid point in the parameter space is employed. However, the way of optimizing the parameters of the evaluation function is not limited to the grid search. In other embodiments, other algorithm including, without limitation, random search, Bayesian optimization and gradient-based optimization can also be employed.

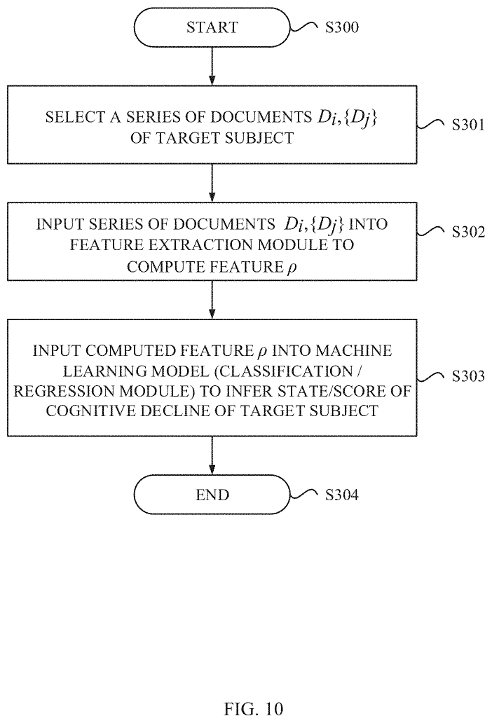

With reference to FIG. 10, a process for detecting a sign of cognitive decline for a subject use using the novel feature ρ according to an exemplary embodiment of the present invention is described. As shown in FIG. 10, the process may begin at step S300 in response to a request for initiating a detection process for a target subject user 114 from his/her family member, for example. Note that the process shown in FIG. 10 may be performed by processing circuitry such as one or more processing units.

At step S301, the processing circuitry may select a series of conversation documents (Dᵢ, {Dⱼ}) of the target subject user 114 that are within an appropriate period.

At step S302, the processing circuitry may input the selected series of the conversation documents (Dᵢ, {Dⱼ}) into the feature extraction module 130 to compute the feature ρ.

In the process shown in step S302, the evaluation value Sᵢⱼ and the topic similarity Yᵢⱼ are calculated for each pair of the reference document Dᵢ and each document in the document set {Dⱼ}. The evaluation value Sᵢⱼ is calculated by the evaluation function with the optimized weights β_{T}*, β_{N}*, β_{P}*, β_{Q}*. The feature ρ is computed from the relationship between the evaluation values Sᵢⱼ and the topic similarities Yᵢⱼ.

At step S303, the processing circuitry may input the computed feature ρ into the machine learning model (e.g., the classification/regression module 140) to infer the state/score of the cognitive decline of the target individual and the process ends at step S304.

Note that, in the inference mode, the machine learning model used to infer the state/score of the cognitive decline may be same as or different from the classification/regression module 140 used to evaluate the discriminative power in the learning mode. For example, the parameters of the feature extraction module 130 are optimized by using a simple binary classifier based solely on the feature ρ in the learning mode. In the inference mode, the feature ρ can be used as an input for other sophisticated machine learning model such as deep neural network in combination with other feature.

According to one or more embodiments of the present invention, the feature suitable for detecting a sign of cognitive decline can be computed from the conversation documents recorded for an individual. The novel feature that characterizes change in topic similarity over conversations on different days of the individual well evaluates potential risks of cognitive decline. Leveraging the specially designed feature can lead a performance improvement for detecting the sign of the cognitive decline. Since the feature shows larger discriminative power, i.e., the distribution of the features for the control group and the distribution of the features for the patient group are separated preferably even simple classifiers that do not require so many computational resources can classify well based on the feature. Enriching of features that can be used to detect the sign of the cognitive decline can reduce the computational resources by way of (1) providing an efficient feature set composed of fewer features and/or (2) providing a model having higher generalization performance to avoid the need for building models individually and specifically designed for each individual and for each situation.

Note that the languages to which the novel feature extraction technique is applicable is not limited and such languages may include, but is not limited to, Arabic, Chinese, English, French, German, Japanese, Korean, Portuguese, Russian, Spanish, for instance.

### Experimental Studies

A program implementing modules 120, 122, 130, 140, 150 of the system indicated by the rectangle with a dashed border in FIG. 1 and the process shown in FIG. 2, FIG. 8, and FIG. 10 according to the exemplary embodiment was coded and executed for given sample documents.

The sample documents were plural sets of daily conversational data obtained from a monitoring service for elderly people. The purpose of this service is to help children to build a connection with their parent living alone by sharing the daily life information of elderly people, such as their physical condition. The human communicator called elderly people once or twice a week to have a daily conversation for about ten minutes. Each conversation was transcribed in spoken word format by the communicator and sent to the family by email as a report. The conversational data were collected from eight Japanese people (five females and three males; age range 66-89 years, i.e., 82.37±5.91 years old). Two of them were reported as suffering from dementia from the family.

All reports were written in Japanese. For preprocessing, linguistic analysis including word segmentation, part-of-speech tagging and word lemmatization on the conversational data were performed. Only words tagged as nouns were used as an input for topic modelling. LDA was employed as topic modeling. L(=K) topics were extracted from each noun set Uₓ by giving each document Uₓ as the corpus D for estimating the LDA topic model. Maximum of cosine similarities between vectors in all combination of L vectors for Dᵢ and L vectors for Dⱼ (L×L similarities) was used as the value of the topic similarity Yᵢⱼ.

As for Examples and Comparative Examples, the proposed feature (Pearson correlation coefficient ρ between the topic similarity Yᵢⱼ and the evaluation value Sᵢⱼ) and other conventional features were investigated using the conversational data obtained during the phone calls with the regular monitoring service. The discriminative power was measured by using both effect size (Cohen's d) and Area Under the Receiver (AUC)-Operating characteristic curve (ROC). For Cohen's d, the 0.8 effect size can be assumed to be large, while the 0.5 effect size is medium and the 0.2 effect size is small. ROC is a graphical plot that illustrates the diagnostic ability of a binary classifier model that ranges from 0 to 1.

As for Example 1, the feature ρ was calculated from sample conversation documents {Dⱼ} recorded within 30 day from a given sample reference document Dᵢ.

The evaluation value Sᵢⱼ was calculated as a weighted sum of all features Tᵢⱼ, Nᵢⱼ, Pᵢⱼ, Qᵢⱼ with hyper-parameters β_{T}*, β_{N}*, β_{P}*, β_{Q}*. The hyper-parameters β_{T}*, β_{N}*, β_{P}*, β_{Q}* were selected by the parameter optimization. To evaluate discriminative power, the feature ρ was calculated for each of possible reference document Dᵢ. As a result, the effect size of −2.63 (95% confidential interval (CI): −3.68, −1.60) and the AUC-ROC of 0.96 were obtained.

As Comparative Examples 1-5, other features extracted from single conversation, including vocabulary richness, sentence complexity, and repetitiveness, were also investigated. As for vocabulary richness, Honore's statistics (Comparative Example 1), Type-Token Ratio (Comparative Example 4) and Brunet's Index (Comparative Example 5) were used. For sentence complexity and repetitiveness, sentence similarity (Comparative Example 2) and mean sentence length (Comparative Example 3) were employed, respectively. The sentence similarity was computed using cosine distance of sentences defined as TF-IDF (Term Frequency-Inverse Document Frequency) vectors.

Among the six features (Example 1, Comparative Examples 1-5), the proposed feature ρ (Example 1) showed the best results in terms of effect size and ROC (d=−2.63, ROC=0.96), followed by Honore's Statistics (Comparative Example 1) (d=−0.98, ROC=0.82), and the sentence similarity (Comparative Example 2) (d=0.42, ROC=0.72). The results are summarized in Table 1.

TABLE 1

| Feature type | Effect Size (95% CI) | AUC-ROC |
|---|---|---|
| Pearson correlation coefficient ρ | −2.63 | 0.96 |
| (Example 1) | (−3.68, −1.60) | |
| Honore's statistics | −0.98 | 0.82 |
| (Comparative Example 1) | (−1.26, −0.70) | |
| Sentence similarity | 0.42 | 0.72 |
| (Comparative Example 2) | (0.15, 0.69) | |
| Mean sentence length | 0.22 | 0.63 |
| (Comparative Example 3) | (−0.05, 0.69) | |
| Type Token Ratio | 0.06 | 0.50 |
| (Comparative Example 4) | (−0.21, 0.33) | |
| Brunet's Index | −0.05 | 0.51 |
| (Comparative Example 5) | (−0.32, 0.22) | |

Since the proposed feature ρ was computed based on the evaluation value Sᵢⱼ that was calculated using all features Tᵢⱼ, Nᵢⱼ, Pᵢⱼ, Qᵢⱼ in the Example 1, the usefulness of combining these feature Tᵢⱼ, Nᵢⱼ, Pᵢⱼ, Qᵢⱼ was also investigated. As for Examples 2 and 3, the number of days between the conversations Tᵢⱼ and the number of documents Nᵢⱼ were used solely as the evaluation value Sᵢⱼ that evaluates at least the temporal separation between conversations corresponding to the reference document Dᵢ and other document Dⱼ, respectively. As for Comparative Examples 6 and 7, instead of using the evaluation value Sᵢⱼ, an amount of speeches spoken by the user Pᵢⱼ and a total amount of speeches Qᵢⱼ, were used solely to compute the Pearson correlation coefficient, respectively. Note that total amount of the speeches and the individual amount of the speeches spoken by the user Pᵢⱼ were measured as the number of the nouns in the combined document Dᵢⱼ.

The proposed feature calculated using the evaluation value Sᵢⱼ that was a function of the four features Tᵢⱼ, Nᵢⱼ, Pᵢⱼ, Qᵢⱼ showed best in comparison with that was calculated using solely one of the features Tᵢⱼ, Nᵢⱼ, Pᵢⱼ, Qᵢⱼ. Note that the type of the features evaluating the amount of the speeches (Pᵢⱼ and Qᵢⱼ) showed opposite effect from the type of the features evaluating the temporal separation (Tᵢⱼ, Nᵢⱼ). The results are summarized in Table 2.

TABLE 2

| Feature type | Effect Size (95% CI) | AUC- ROC |
|---|---|---|
| Pearson correlation coefficient ρ | −2.63 | 0.96 |
| using the evaluation value Sᵢⱼ = | (−3.68, −1.60) | |
| β_{N} Tᵢⱼ + β_{T} Nᵢⱼ − β_{P} Pᵢⱼ − β_{Q} Qᵢⱼ | | |
| (Example 1: Same as Table 1) | | |
| Pearson correlation coefficient ρ | −2.45 | 0.92 |
| using evaluation value Sᵢⱼ = Tᵢⱼ | (−3.48, −1.43) | |
| (Example 2) | | |
| Pearson correlation coefficient ρ | −2.26 | 0.85 |
| using evaluation value Sᵢⱼ = Nᵢⱼ | (−3.27, −1.25) | |
| (Example 3) | | |
| Pearson correlation coefficient | 1.31 | 0.82 |
| using evaluation value Sᵢⱼ = Pᵢⱼ | (0.35, 2.26) | |
| (Comparative Example 6) | | |
| Pearson correlation coefficient | 1.47 | 0.79 |
| using evaluation value Sᵢⱼ = Qᵢⱼ | (0.50, 2.43) | |
| (Comparative Example 7) | | |

As described above, it was found that the proposed feature ρ has strong discriminating power and achieved up to −2.63 for effect size of Cohen's d and 0.96 for AUC-ROC scores. It was also demonstrated that the proposed feature ρ outperformed other conventional features, suggesting that the use of the proposed feature ρ in addition to the conventional features has promise to improve detection performance. It was also shown that the proposed features p calculated by using the evaluation value combining the features Tᵢⱼ, Nᵢⱼ, Pᵢⱼ, Qᵢⱼ may be more advantageous in enhancing discriminative power than the features calculated by using solely one of the features Tᵢⱼ, Nᵢⱼ, Pᵢⱼ, Qᵢⱼ.

Computer Hardware Component

Referring now to FIG. 11, a schematic of an example of a computer system 10, which can be used for the diagnosis support system 100, is shown. The computer system 10 shown in FIG. 11 is implemented as computer system. The computer system 10 is only one example of a suitable processing device and is not intended to suggest any limitation as to the scope of use or functionality of embodiments of the invention described herein. Regardless, the computer system 10 is capable of being implemented and/or performing any of the functionality set forth hereinabove.

The computer system 10 is operational with numerous other general purpose or special purpose computing system environments or configurations. Examples of well-known computing systems, environments, and/or configurations that may be suitable for use with the computer system 10 include, but are not limited to, personal computer systems, server computer systems, thin clients, thick clients, hand-held or laptop devices, in-vehicle devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system 10 may be described in the general context of computer system-executable instructions, such as program modules, being executed by a computer system. Generally, program modules may include routines, programs, objects, components, logic, data structures, and so on that perform particular tasks or implement particular abstract data types.

As shown in FIG. 11, the computer system 10 is shown in the form of a general-purpose computing device. The components of the computer system 10 may include, but are not limited to, a processor (or processing unit) 12 and a memory 16 coupled to the processor 12 by a bus including a memory bus or memory controller, and a processor or local bus using any of a variety of bus architectures.

The computer system 10 typically includes a variety of computer system readable media. Such media may be any available media that is accessible by the computer system 10, and it includes both volatile and non-volatile media, removable and non-removable media.

The memory 16 can include computer system readable media in the form of volatile memory, such as random access memory (RAM). The computer system 10 may further include other removable/non-removable, volatile/non-volatile computer system storage media. By way of example only, the storage system 18 can be provided for reading from and writing to a non-removable, non-volatile magnetic media. As will be further depicted and described below, the storage system 18 may include at least one program product having a set (e.g., at least one) of program modules that are configured to carry out the functions of embodiments of the invention.

Program/utility, having a set (at least one) of program modules, may be stored in the storage system 18 by way of example, and not limitation, as well as an operating system, one or more application programs, other program modules, and program data. Each of the operating system, one or more application programs, other program modules, and program data or some combination thereof, may include an implementation of a networking environment. Program modules generally carry out the functions and/or methodologies of embodiments of the invention as described herein.

The computer system 10 may also communicate with one or more peripherals 24 such as a keyboard, a pointing device, a car navigation system, an audio system, etc.; a display 26; one or more devices that enable a user to interact with the computer system 10; and/or any devices (e.g., network card, modem, etc.) that enable the computer system 10 to communicate with one or more other computing devices. Such communication can occur via Input/Output (I/O) interfaces 22. Still yet, the computer system 10 can communicate with one or more networks such as a local area network (LAN), a general wide area network (WAN), and/or a public network (e.g., the Internet) via the network adapter 20. As depicted, the network adapter 20 communicates with the other components of the computer system 10 via bus. It should be understood that although not shown, other hardware and/or software components could be used in conjunction with the computer system 10. Examples, include, but are not limited to: microcode, device drivers, redundant processing units, external disk drive arrays, RAID systems, tape drives, and data archival storage systems, etc.

Computer Program Implementation

The present invention may be a computer system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more aspects of the present invention has been presented for purposes of illustration and description, but is not intended to be exhaustive or limited to the invention in the form disclosed.

Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for supporting detection of a sign of cognitive decline, the method comprising:
obtaining a reference set of conversational data recorded for an individual and one or more sets of conversational data recorded for the individual on different days from the reference set;
evaluating at least a temporal separation between conversations corresponding to the reference set and each of the one or more sets of the conversational data to determine a value of the temporal separation;
determining topic similarity between the reference set and each of the one or more sets of the conversational data;
generating a feature for the individual based, at least in part, on relationship between the value and the topic similarity; and
sending a message corresponding to a diagnosis by the feature computed for the individual.

2. The method of claim 1, wherein the value is calculated by further evaluating an amount of speeches in the conversations corresponding to the reference set and each of the one or more sets of the conversational data.

3. The method of claim 2, wherein the temporal separation is evaluated by the number of days between conversations corresponding to the reference set and the each of the one or more sets or the number of sets of conversational data existing between the reference set and the each of the one or more sets, and the amount of the speeches is evaluated by an amount of speeches spoken by the individual included in both the reference set and each of the one or more sets or a total amount of speeches included in both the reference set and each of the one or more sets.

4. The method of claim 2, wherein the value is calculated as a weighted sum of one or more features evaluating the temporal separation and one or more features evaluating the amount of the speeches, with corresponding weights.

5. The method of claim 4, wherein the weights used for calculating the value are optimized by:
preparing one or more training samples each including one or more sample sets of conversational data recorded for a participant and a label regarding the cognitive decline;
setting provisional values for the weights;
computing a trial result of the feature under the provisional values of the weights by using the one or more training samples;
evaluating discriminative power using the trial result of the feature, the discriminative power being evaluated by using a corresponding label in the one or more training samples; and
finding optimal values for the weights based on the discriminative power.

6. The method of claim 1, wherein the feature is a correlation coefficient between the topic similarity and the value evaluating at least the temporal separation.

7. The method of claim 1, wherein the topic similarity is calculated based on Latent Dirichlet Allocation (LDA).

8. The method of claim 1, wherein the calculating the topic similarity comprises:
performing linguistic analysis on each of the reference set and the one or more sets to obtain a reference noun set for the reference set of the conversational data and one or more noun sets for the one or more sets of the conversational data;
extracting one or more topics from each of the reference noun set and the one or more noun sets to obtain a reference topic set and one or more topic sets; and
calculating similarity between the reference topic set and each of the one or more topic sets.

9. The method of claim 1, wherein the individual is a target for inference and the feature calculated for the individual is used as an input for a machine learning model solely or in combination with other feature to infer whether or not there is the sign of the cognitive decline, or the degree of the risk of the cognitive decline.

10. The method of claim 1, wherein the individual is a participant associated with a label regarding the cognitive decline and the feature calculated for the individual is used as an input for a machine learning model solely or in combination with other feature to optimize parameters for inference.

11. A computer system for supporting detection of a sign of cognitive decline, by executing program instructions, the computer system comprising:
a memory tangibly storing the program instructions;
a processor in communications with the memory, wherein the processor is configured to:
obtain a reference set of conversational data recorded for an individual and one or more sets of conversational data recorded for the individual on different days from the reference set;
evaluating at least a temporal separation between conversations corresponding to the reference set and each of the one or more sets of the conversational data to determine a value of the temporal separation;
determine topic similarity between the reference set and each of the one or more sets of the conversational data;
generate a feature for the individual based, at least in part, on relationship between the value and the topic similarity; and
sending a message corresponding to a diagnosis by the feature computed for the individual.

12. The computer system of claim 11, wherein the value is calculated by further evaluating an amount of speeches in the conversations corresponding to the reference set and each of the one or more sets of the conversational data.

13. The computer system of claim 12, wherein the temporal separation is evaluated by the number of days between conversations corresponding to the reference set and the each of the one or more sets or the number of sets of conversational data existing between the reference set and the each of the one or more sets, and the amount of the speeches is evaluated by an amount of speeches spoken by the individual included in both the reference set and each of the one or more sets or a total amount of speeches included in both the reference set and each of the one or more sets.

14. The computer system of claim 12, wherein the value is calculated as a weighted sum of one or more features evaluating the temporal separation and one or more features evaluating the amount of the speeches, with corresponding weights.

15. The computer system of claim 14, wherein the weights used for calculating the value are optimized by using one or more training samples each including one or more sample sets of conversational data recorded for a participant and a label regarding the cognitive decline.

16. The computer system of claim 11, wherein the feature is a correlation coefficient between the topic similarity and the value evaluating at least the temporal separation.

17. The computer system of claim 11, wherein the individual is a target for inference and the feature calculated for the individual is used as an input for a machine learning model solely or in combination with other feature to infer whether or not there is the sign of the cognitive decline, or the degree of the risk of the cognitive decline.

18. The computer system of claim 11, wherein the individual is a participant associated with a label regarding the cognitive decline and the feature calculated for the individual is used as an input for a machine learning model solely or in combination with other feature to optimize parameters for inference.

19. A computer program product for supporting detection of a sign of cognitive decline, the computer program product comprising a computer readable storage medium having program instructions embodied therewith, the program instructions executable by a computer to cause the computer to perform a method, the method comprising:
obtaining a reference set of conversational data recorded for an individual and one or more sets of conversational data recorded for the individual on different days from the reference set;
evaluating at least a temporal separation between conversations corresponding to the reference set and each of the one or more sets of the conversational data to determine a value of the temporal separation;
determining topic similarity between the reference set and each of the one or more sets of the conversational data;
generating a feature for the individual based, at least in part, on relationship between the value and the topic similarity; and
sending a message corresponding to a diagnosis by the feature computed for the individual.

20. The computer program product of claim 19, wherein the value is calculated by further evaluating an amount of speeches in the conversations corresponding to the reference set and each of the one or more sets of the conversational data.
